(19) United States
(12) Patent Application Publication
(10) Pub. No.: US 2022/0064585 A1
(43) Pub. Date: Mar. 3, 2022

(54) DEVICE FOR BREAKING CELLS

(71) Applicant: GRIFOLS DIAGNOSTIC SOLUTIONS INC., EMERYVILLE, CA (US)

(72) Inventors: HELCIO BURD, BERKELEY, CA (US); THOMAS STAPP, SAN FRANCISCO, CA (US); YUYI SHEN, DANVILLE, CA (US)

(21) Appl. No.: 17/420,070

(22) PCT Filed: Jan. 9, 2020
(86) PCT No.: PCT/IB2020/050146

§ 371 Date: Jun. 30, 2021

Related U.S. Application Data
(60) Provisional application No. 62/791,190, filed on Jan. 11, 2019.

(51) Int. Cl. C12M 1/00; C12M 1/02

(52) U.S. Cl. CPC C12M 47/06; C12M 41/22

(57) ABSTRACT

A device for breaking cells that has a reactor with a reaction chamber, an agitator, a cooling jacket, a cooling jacket inlet, a cooling jacket outlet, a sampling port, and a temperature probe insertion fitting, and a motor suitable for mounting the reactor on its top and which is operably connected with the agitator. A system for breaking cells comprising at least one device, at least one temperature probe inserted in the temperature probe insertion fitting of the device, a cooling system operably connected to the temperature probe, and an electronic control panel. The cooling system has at least one solenoid valve, each device of the system having one corresponding solenoid valve.

### PRIORITY AND CROSS REFERENCE TO RELATED APPLICATIONS

This application is the U.S. National Phase Application under 35 U.S.C. § 371 of International Application No. PCT/IB2020/050146, filed Jan. 9, 2020, designating the U.S. and published in English as WO 2020/144616 A1 on Jul. 16, 2020, which claims the benefit of U.S. Provisional Application No. 62/791,190, filed Jan. 11, 2019. Any and all applications for which a foreign or a domestic priority is claimed is/are identified in the Application Data Sheet filed herewith and is/are hereby incorporated by reference in their entireties under 37 C.F.R. § 1.57.

### FIELD

The present invention refers to the biotechnology field, and in particular it refers to a device for cell disruption which provides very accurate temperature control with data recording and with the ability to sample the cells during processing.

### BACKGROUND

Cell disruption is the method or process for releasing biological molecules from inside a cell. Utilizing intracellular contents such as proteins, organelles, DNA/RNA, and enzymes found and/or grown inside cells has become a new generation of drug and diagnostic tools development. Many biotechnologically produced compounds are intracellular and must be released from cells before recovery. The efficient recovery of said products requires cell disruption, which can be achieved by using different methods and technologies, either mechanical or non-mechanical methods. The chosen technology depends on the product, cell type and scale. The cell disruption mechanical methods which are commonly used include the bead mill, sonication and French press. Other possible methods are the utilization of enzymes, detergents and osmotic shock.

### SUMMARY

In some embodiments, a device for breaking cells that has a reactor with a reaction chamber, an agitator, a cooling jacket, a cooling jacket inlet, a cooling jacket outlet, a sampling port, and a temperature probe insertion fitting, and a motor suitable for mounting the reactor on its top and which is operably connected with the agitator. A system for breaking cells comprising at least one device, at least one temperature probe inserted in the temperature probe insertion fitting of the device, a cooling system operably connected to the temperature probe, and an electronic control panel. The cooling system has at least one solenoid valve, each device of the system having one corresponding solenoid valve.

### BRIEF DESCRIPTION OF THE DRAWINGS

FIG. 1 shows a perspective view of a partially crossed embodiment of the reactor of the device for breaking cells of the present invention.

FIG. 2 is a perspective view of an embodiment of the reactor of the device for breaking cells of the present invention.

FIG. 3 shows a perspective view of the reactor of the device for breaking cells of the present invention on top of the motor drive.

FIG. 4 shows a perspective view of four devices for breaking cells according to the present invention with their correspondent solenoid valves.

FIG. 5 is a schematic view of the cooling system in the inactive (not cooling) state.

FIG. 6 is a schematic view of the cooling system in the active (cooling) state.

FIG. 7 is a graph with data logged during a cell lysis process using the device of the present invention.

FIG. 8 is a graph of the release HBCORE protein determined by gel measurements during a cell breaking process.

### DETAILED DESCRIPTION

Bead mills have been originally used in the paint industry, and have been adapted for cell disruption in both small scale and large scale production. It is an efficient way of disrupting different microbial cells as different designs have been developed. The main principle requires a jacketed grinding chamber with a rotating shaft, running in its center. Agitators are fitted with the shaft, and provide kinetic energy to the small beads that are present in the chamber, making the beads collide with each other. The choice of bead size and weight is greatly dependent on the type of cells. The bead diameter can affect the efficiency of cell disruption in relation of the location of the desired enzyme in the cell. The increased number of beads increases the degree of disruption, due to the increased bead-to-bead interaction. The increased number of beads, however, also affects the heating and power consumption. The process variables are: agitator speed, proportion of the beads, beads size, cell suspension concentration, cell suspension flow rate, and agitator disc design.

Main issues related to bead mills, are the high temperature rises with increase of bead volume. These conditions would affect protein release, protein solubility and cause undesirable effects in the products.

Usually, the existing bead mill devices have uncontrolled cooling and neither allows sampling, acquisition or control of temperature during the cell lysis process. The present inventors have developed a cell breaking device that overcome all the drawbacks of the prior art devices. The cell disruptor of the present invention provides accurate temperature control and recording, using feedback between a temperature probe in the reactor chamber and the flow of cooling liquid jacketing the chamber, achieving a more accurate assessment of the effect of process conditions on cell breakage as compared with the previous devices. Improper temperature control leads to product degradation and an erroneous assessment of the effect of the glass bead/cell collisions. Another feature included in the cell breaking device of the present invention is the possibility of removal of sample material to determine breakage without interrupting the process, thus obtaining samples during the cell disruption process, which allows information to be gained regarding the efficiency of the process at intermediate times. All these features in combination give a more accurate assessment of the cell disruption process, and result in a decreased time of process and an increased reproducibility, for example.

As used herein the terms cell “breaking”, “disruption”, and “lysis” are interchangable and they mean the same and refer to the breaking down of the membrane of a cell.

To aid understanding, the present invention is described in greater detail below, with reference to the attached figures, which are presented by way of example, and with reference to illustrative but nonlimiting examples.

In a first aspect, the present invention refers to a device for breaking cells comprising:

a reactor comprising a reaction chamber, an agitator, a cooling jacket, a cooling jacket inlet, a cooling jacket outlet, a sampling port, and a temperature probe insertion fitting;
a motor suitable for mounting said reactor on its top and which is operably connected with said agitator.

Preferably, the volume of said reaction chamber is between 250 mL and 600 mL, more preferably between 300 mL and 500 mL.

In another aspect, the present invention refers to a system for breaking cells using the above-described device comprising:

at least one device for breaking cells as mentioned above;
at least one temperature probe inserted in the temperature probe insertion fitting of said device for breaking cells;
a cooling system operably connected to said temperature probe; and
an electronic control panel.

Preferably, said system comprises at least two devices for breaking cells in parallel, more preferably three, and the most preferably four devices for breaking cells in parallel.

Preferably the cooling system comprise at least one solenoid valve, more preferably each device for breaking cell has one solenoid valve. Also preferably said cooling system uses chilled water as cooling fluid.

With the system using the device for breaking cells of the present invention is possible to run several disruption process in parallel, for example four, using different cells, buffers, temperature set point and processing times.

The system of the present invention uses a closed temperature loop control. For example, the motor would stop when the temperature gets 1° C. above the set point (SP). This allows heat generation to stop (pausing the cell breakage) and only heat removal happens until the temperature reaches 1° C. below the SP and the motor starts again (continuing the breakage). This closed loop control is very important for batch runs because the cells suspension is always within the reactor and if the temperature gets high there will be product degradation and the entire suspension within the reactor will be lost.

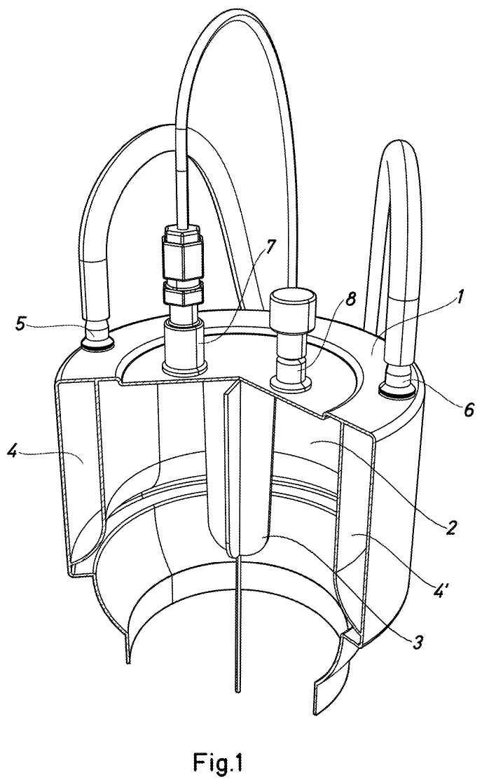

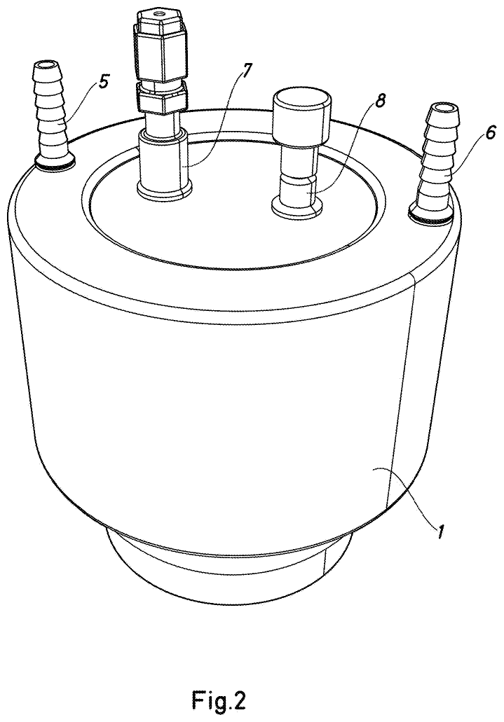

FIG. 1 shows a perspective view of a partially crossed embodiment of the reactor of the device for breaking cells of the present invention. Said reactor -1- comprises a reaction chamber -2-, an agitator -3-, a cooling jacket -4-, -4′-, a cooling jacket inlet -5-, a cooling jacket outlet -6-, a sampling port -7-, and a temperature probe insertion fitting -8-. As shown in FIG. 2, said reaction chamber is a closed space configured to receive a mixture of bead mills and the cell suspension to be disrupted, and only the sampling port -7- and the temperature probe inserted in the temperature probe insertion fitting -8- are fluidly connected with the reaction chamber -2-.

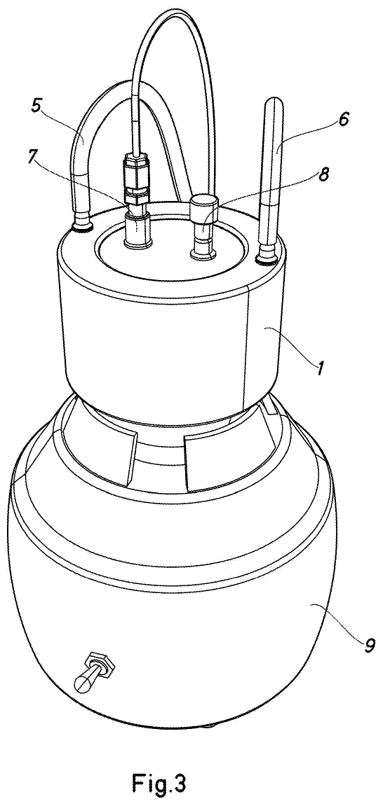

As shown in FIG. 3, the reactor -1- is coupled to an electric motor -9- on its top. Said electric motor -9- is operably connected to the agitator -3- of the reactor -1- and its functioning is controlled by the cooling system, which turn the motor -9- on or off depending on the temperature variability with respect to the set point.

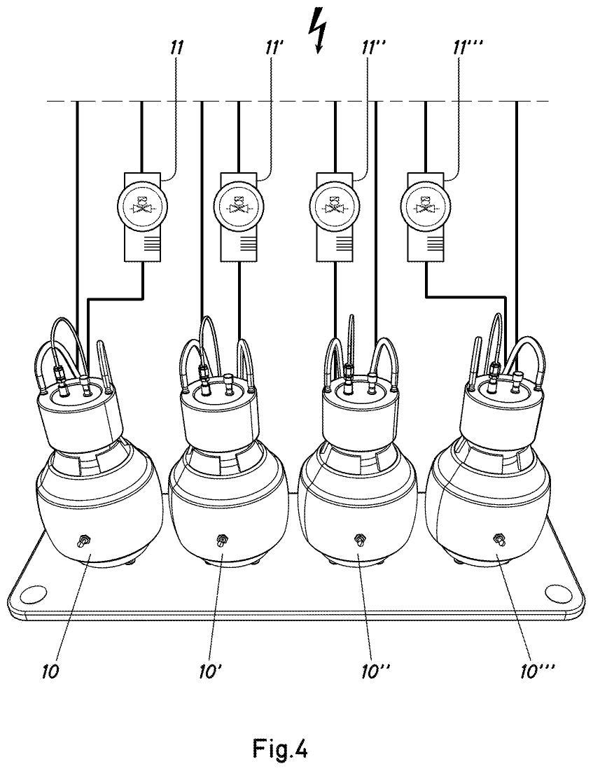

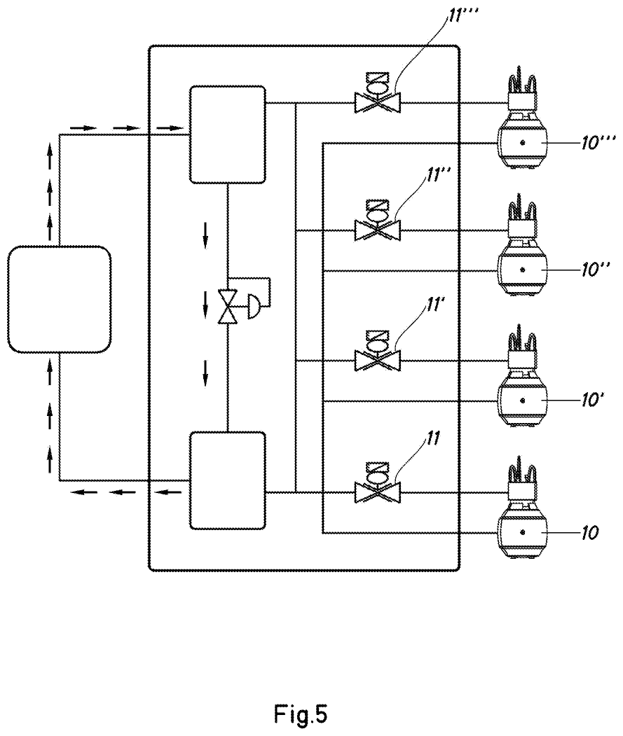

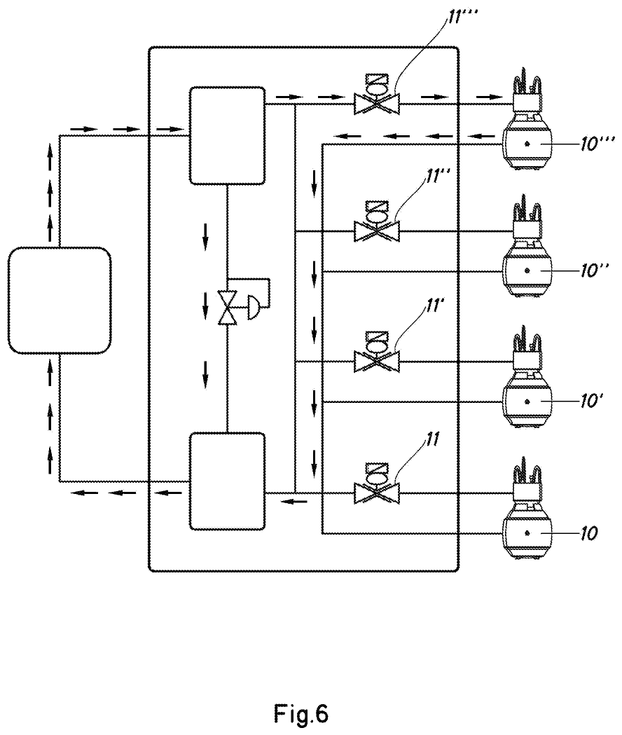

FIG. 4 shows shows four devices -10-, -10′-, -10″-, -10′″- for breaking cells according to the present invention, with their correspondent solenoid valves -11-, -11′-, -11″-, -11′″-. FIG. 5 shows a diagram of the automated chilled water distribution when the all solenoid valves -11-, -11′-, -11″-, -11′″- are turned off. The black arrows indicate the direction of the water flow. On the other hand, FIG. 6 shows a similar diagram than FIG. 5 but when only one solenoid valve -11′″- is turned on. Again the black arrows indicate the direction of the water flow.

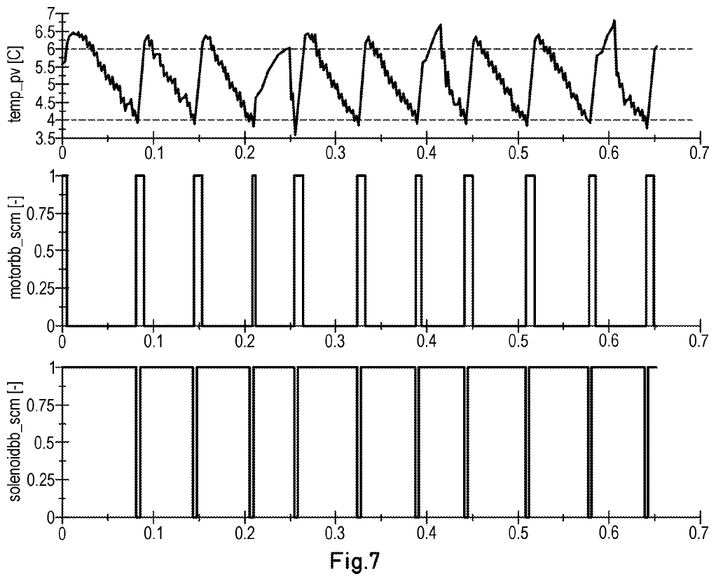

FIG. 7 shows data logged during the cell lysis process using a device for breaking cells of the present invention. The top graph shows the temperature, the middle graph shows the motor status (1=On/0=Off) and the bottom graph shows the solenoid valve status (1=On/0=Off) within the chilled water distribution system. It can be seen that when the motor is on, the solenoid valve is turned off and the temperature increases. On the contrary, when said temperature is above 6° C. (set point) the motor is turn off, the solenoid valve is turn on and the temperature decreases. This cycle is repeated until the desired degree of cell lysis is achieved.

Hereinafter, the present invention is described with reference to examples, which however are not intended to limit the present invention.

### EXAMPLE 1

### Cell Disruption Using the Cell Breaking Device of the Present Invention

Two devices of the present invention were filled with 280 mL of glass beads and 200 mL of BYS (buffer plus about 40 g of cells) in each of them. The temperature set point for the fluid inside the reactor chamber was 5° C. and the dead band was set to 2° C. for both cell breaking devices in the control software. The cooling fluid (Glycol) temperature was set to −15 C at the exit of the chiller. Samples for HBCORE released quantification were collected (using the device sampling port) during 5 min at regular intervals. The released HBCORE curve was determine using SDS-PAGE gel electrophoresis.

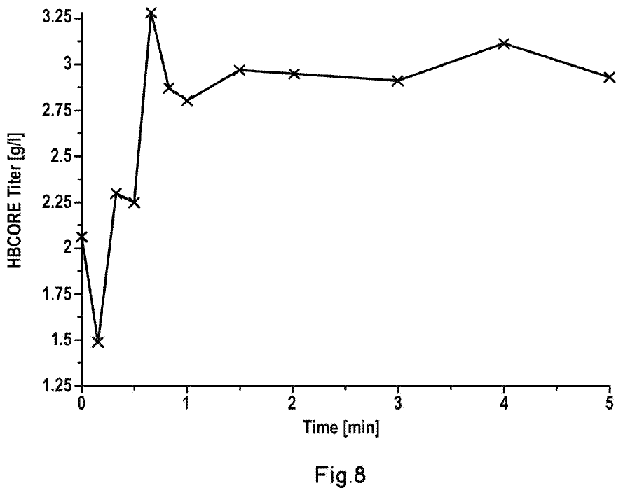

As can be seen in FIG. 8, release of HBCORE protein reaches a plateau at about 1 min and further processing after this time does not contribute to increase the HBCORE concentration. This time point can be considered as the Maximum Breakage Point. At this time process should be stopped.

### EXAMPLE 2

### Cell Disruption Using a Cell Breaking Device of the Prior Art

A bead mill commercialized with the trade name Dyno-Mill KD6 (Eskens, The Netherlands) was used as a cell disruption device. This grinding mill is available with grinding chamber volumes from 6 L. In this case a Dyno-Mill with 6 L of grinding chamber was used. This bead mill is used in continuous mode due to the large amount of cells to be processed. For that, a vessel (input vessel) containing the 1 L of BYS (the same cells as in Example 1) was connected to the Dyno-Mill and a peristaltic pump was added between these two. A second vessel (output vessel) was used to collect the output at the same time that the BYS from the input vessel is pumped into the Dyno-Mill. The output vessel, which was kept on ice, was swapped with the input vessel when it becomes empty to start a second pass. The glass beads, which occupy about 85% by volume of the reaction chamber, are retained inside the Dyno-Mill at all time due to a special mechanism that avoids the glass beads getting out the reactor chamber. The reactor chamber was cooled as usually, with glycol at −12° C., flowing through its cooling jacket continuously during the entire breaking process. The flow rate for the peristaltic pump was set to 100 mL/min. As a result, the same cell disruption degree as in Example 1 (1 min) was obtained after two passes (approx. 20 min), regardless the time needed to wash the beads at the end of the process (further 5 min).

1. A device for breaking cells comprising:
a) a reactor comprising a reaction chamber, an agitator, a cooling jacket, a cooling jacket inlet, a cooling jacket outlet, a sampling port, and a temperature probe insertion fitting;
b) a motor suitable for mounting said reactor on its top and which is operably connected with said agitator.

2. The device according to claim 1, wherein the volume of said reaction chamber is between 100 mL and 600 mL.

3. The device according to claim 2, wherein the volume of said reaction chamber is between 300 mL and 500 mL.

4. A system for breaking cells, comprising:
a) at least one device for breaking cells according to claim 1;
b) at least one temperature probe inserted in the temperature probe insertion fitting of said at least one device for breaking cells;
c) a cooling system operably connected to said temperature probe; and
d) an electronic control panel.

5. The system according to claim 4, wherein said system comprises at least two devices for breaking cells in parallel.

6. The system according to claim 5, wherein said system comprises four devices for breaking cells in parallel.

7. The system according to claim 4, wherein said cooling system comprise at least one solenoid valve.

8. The system according to claim 4, wherein each device for breaking cell has one corresponding solenoid valve.

9. The system according to claim 4, wherein said cooling system comprises a cooling fluid.

10. The system according to claim 9, wherein the cooling fluid is glycol.

11. The system according to claim 9, wherein a temperature of the cooling fluid is −15 C.
